(19) United States
(12) Patent Application Publication
(10) Pub. No.: US 2017/0296510 A1
(43) Pub. Date: Oct. 19, 2017

(54) METHOD OF FORMING CONCENTRATED SOLUTION

(71) Applicant: Prosonix Limited, Oxford (GB)

(72) Inventors: John Burns, Oxford (GB); Dipesh Parikh, Oxford (GB)

(21) Appl. No.: 15/513,923

(22) PCT Filed: Sep. 24, 2015
(86) PCT No.: PCT/GB2015/052763

§ 371 Date: Mar. 23, 2017

(30) Foreign Application Priority Data
Sep. 25, 2014 (GB) 1416909.8

(51) Int. Cl. A61K 31/40; A61K 45/06; A61K 31/138; A61K 9/00; A61K 9/08; A61K 47/10; A61K 31/167

(52) U.S. Cl. CPC A61K 31/40; A61K 47/10; A61K 45/06; A61K 31/167; A61K 9/0078; A61K 9/08; A61K 9/008; A61K 31/138

(57) ABSTRACT

The present invention relates to a method of forming a concentrated solution of first and second pharmacologically active ingredients which involves: providing a solid eutectic composition of the first and second pharmacologically active ingredients; providing a first solvent; and dissolving the eutectic composition in the first solvent; wherein the first and second pharmacologically active ingredients are independently selected from β2 agonists, muscarinic antagonists, anticholinergics, corticosteroids, methylxanthine compounds and salts, esters, polymorphs, hydrates or solvates thereof. A solution obtainable by this method is also described, along with an ampoule for a nebuliser and a pressurised metered dose inhaler comprising the solution. The solution is useful in the treatment of respiratory diseases.

### FIELD OF THE INVENTION

The present invention relates to a method of forming a concentrated solution of first and second pharmacologically active ingredients which involves providing a solid eutectic composition of the first and second pharmacologically active ingredients and dissolving this in a solvent. The solution is useful in the treatment of respiratory diseases.

### BACKGROUND OF THE INVENTION

A simple eutectic composition consists of two compounds which are completely miscible in the liquid state but only to a very limited extent in the solid state. The unique property of a eutectic is that it has a lower melting temperature than that of either of the pure compounds. Eutectics have many of the same properties as each phase, but behave differently from either component with respect to melting point, solubility and chemical stability. Eutectic compositions are known in a wide variety of medical fields. WO 2011/014850 discloses forming a eutectic liquid, and then adding a solvent to make a highly viscous solution. The formulations are used as topical compositions.

In WO2013/021199 we described the use of solid eutectic compositions for the treatment of respiratory diseases. However, to the best of the inventors' knowledge there is no previous disclosure of using concentrated liquid eutectic compositions in the treatment of respiratory diseases. Further, there is no disclosure of using these eutectic compositions in solution form via pressurized metered dose inhalers, or nebulizers wherein the composition is inhaled into the lung.

Inhalation represents a very attractive, rapid and patient-friendly route for the delivery of systemically acting drugs, as well as for drugs that are designed to act locally on the lungs themselves, such as to treat respiratory diseases, preferably infection or chronic respiratory diseases for example asthma, chronic obstructive pulmonary disease and cystic fibrosis. Drugs can be delivered by inhalation using nebulizers, metered dose inhalers, or dry powder inhalers, which are all well known in the art.

WO2010/144628 provides methods of treating a patient having chronic obstructive pulmonary disease (COPD) comprising administering to the patient, with a high efficiency nebulizer, a long acting beta 2-agonist (LABA). Combinations of LABAs and long acting muscarinic antagonists (LAMAs) are also disclosed, and these may be formulated as solutions which further comprise at least one excipient or active adjunct.

U.S. Pat. No. 7,985,766 further describes a method for treating COPD or asthma comprising administration of a combination of R,R-glycopyrrolate and formoterol. The combination may be administered in the form of a nebulizable composition comprising a dispersion of the active ingredient in an aqueous or organic medium.

Active ingredient formoterol is difficult to store in a sufficiently stable manner in solution to guarantee pharmaceutical quality over lengthy periods of time. For this reason, powders of formoterol have often been used in the past.

EP1683518 relates to a pharmaceutical formulation of formoterol-containing propylene glycol solution for use in inhalation therapy. The solution is stable and does not require addition of preservatives. Formoterol is present between 0.001 and 0.06% and the solution is stored in an ampoule, which may be mixed with purified water or saline for insertion into a nebulizing chamber.

U.S. Pat. No. 6,150,418 relates to propellant-free, active substance concentrate suitable for storage containing formoterol, for use in inhalation or nasal therapy. The formoterol is in the form of its free-base.

WO 2011/076843 relates to pharmaceutical aerosol formulations for use with pressurised metered dose inhalers (PMDIs) comprising glycopyrronium bromide and formoterol or a salt thereof. The formulation is dissolved in HFA propellant and a co-solvent, and additionally comprises an inorganic acid as a stabilizing agent.

As detailed above, it is known to deliver two pharmacologically active ingredients to the lung simultaneously. For example, Advair and Symbicort co-deliver a bronchodilator and a corticosteroid, and therapeutics are known whereby an anticholinergic, such as glycopyrronium bromide, and a bronchodilator, such as indacaterol are administered together. However, the prior art does not teach how to prepare and preserve a concentrated solution of pharmacologically active ingredients selected from β2 agonists, muscarinic antagonists, anticholinergics, corticosteroids, methylxanthine compounds, and salts, esters, polymorphs, hydrates or solvates thereof in a suitable form as a concentrated solution that retains the stability of the pharmacologically active ingredients. The prior art furthermore does not teach how to do this without any added stabilizing or buffering agents, and in a form suitable for mixing with diluents such as water, saline solution, hydrofluoroalkane propellants (e.g. HFA 134 or HFA 227,) or other diluents suitable for use in nebulizer or solution PMDIs.

### SUMMARY OF THE INVENTION

The first aspect of the invention provides a method of forming a solution of a first and second pharmacologically active ingredient comprising:
providing a solid eutectic composition of the first and second pharmacologically active ingredients;
providing a first solvent; and
dissolving the eutectic composition in the first solvent;

wherein the first and second pharmacologically active ingredients are independently selected from β2 agonists, muscarinic antagonists, anticholinergics, corticosteroids, methylxanthine compounds, and salts, esters, polymorphs, hydrates or solvates thereof.

The solution obtained from the first aspect of the invention may be diluted with a second solvent to obtain a final solution. The second aspect of the invention provides an ampoule for use with a nebuliser, comprising the solution or final solution obtained according to the first aspect of the invention.

The third aspect of the invention provides a pressurised metered dose inhaler comprising the solution or final solution obtained according to the first aspect of the invention and a propellant.

The fourth aspect of the invention provides a solution or final solution obtained according to the first aspect of the invention, for use in the treatment of respiratory diseases.

The fifth aspect of the invention provides a method of treatment comprising administering to a patient a solution or final solution obtained according to the first aspect of the invention.

The sixth aspect of the invention provides a solution of a first and second pharmacologically active ingredient in a first solvent, wherein the first and second pharmacologically active ingredients are independently selected from β2 agonists, muscarinic antagonists, anticholinergics, corticosteroids, methylxanthine compounds, and salts, esters, polymorphs, hydrates or solvates thereof, wherein the concentration of first pharmacologically active ingredient is at least 0.0005% w/w and the concentration of the second pharmacologically active ingredient is at least 0.0005% w/w.

The seventh aspect of the invention provides a solution or final solution made by the first aspect of the invention or a solution according to the sixth aspect of the invention for use in a nebuliser or a pressurised metered dose inhaler.

There exists a number of problems of reliably delivering active ingredients to the lung. Some of these were outlined in our previous application WO2013/021199. It is difficult to dissolve powders of active ingredients to create the desired concentration for use in a nebulizer. Furthermore such solutions, once created, are often unstable. The inventors have found that forming an initial concentrated solution of a eutectic composition and then diluting this further when needed for use in an inhalation device allows formation of more concentrated and more stable solutions. This advantageously means that no additional ingredients such as stabilisers, buffers or excipients are required, and that overall less solution needs to be used, leading to greater efficacy of delivery of active ingredients to the lung and better patient compliance.

### DETAILED DESCRIPTION OF INVENTION

In a eutectic composition the two pharmacologically active ingredients materials are independently crystalline. A simple eutectic composition consists of two compounds which are completely miscible in the liquid state but only to a very limited extent in the solid state. Eutectics have many of the same properties as each phase, but behave differently from either component with respect to melting point, solubility and chemical stability. In particular, a eutectic composition has a lower melting point than either of the two pharmacologically active ingredients. A eutectic composition is an intimate mixture of two pharmacologically active ingredients. The method of the invention involves formation of an initial concentrated solution. As a consequence of the mutual lowering of the melting points of the respective drug substances in a eutectic there is reduced thermodynamic stability of each drug leading to an increase in both equilibrium solubility and the rate of dissolution of both drugs when the solution is formed. Starting with a eutectic composition of the at least two pharmacologically active ingredients, it has been found that these can be dissolved into a much reduced volume of solvent, than required if the same amount of a blend of the at least two pharmacologically active ingredients were dissolved.

The pharmaceutical compositions comprising a eutectic composition of two pharmacologically active ingredients of the present invention have advantages in treatment of respiratory diseases. These advantages include improved efficacy of the pharmacologically active ingredients, improvements in the delivery of both the pharmacologically active ingredients to the same area in the lung, or the whole of the area of the lung and improved onset time for the pharmacologically active ingredients.

The two pharmacologically active ingredients may be selected from different classes of agents. The two pharmacologically active ingredients may be selected from the same class of agents.

The first pharmacologically active ingredient is preferably a β2 agonist or salt, ester, polymorph, hydrate or solvate thereof. The second pharmacologically active ingredient is preferably an anticholinergic agent, most preferably a muscarinic antagonist or salt, ester, polymorph, hydrate or solvate thereof.

The β2 agonist is typically selected from the group consisting of formoterol, salmeterol, carmoterol, indacaterol, vilanterol, arformoterol, bambuterol, isoproterenol, milveterol, clenbuterol, olodaterol, fenoterol, salbutamol, levalbuterol, procaterol, terbutaline, pirbuterol, procaterol, metaproterenol, bitolterol, or ritodrine, albuterol and salts, esters, polymorphs, hydrates or solvates. The β2 agonist is generally a long acting β2 agonist (LABA), preferably selected from the group consisting of salmeterol or formoterol and salts, esters, polymorphs, hydrates or solvates thereof. Alternatively the β2 agonist may be a short acting β2 agonist (SABA) such as salbutamol sulphate.

The muscarinic antagonist may be selected from the group consisting of tiotropium, ipratropium, aclidinium, darotropium, glycopyrrolate or umeclidinium and salts, esters, polymorphs, hydrates or solvates thereof. The muscarinic antagonist is generally a long acting muscarinic antagonist (LAMA), preferably selected from the group consisting of glycopyrrolate and tiotropium, and salts, esters, polymorphs, hydrates or solvates thereof. Alternatively, the muscarinic antagonist is a short acting muscarinic antagonist (SAMA) such as Ipratropium bromide.

Alternatively the active ingredient is a corticosteroid. Preferred corticosteroids are selected from the group consisting of mometasone, beclomethasone, budesonide, fluticasone, ciclesonide or triamcinolone and salts, esters, polymorphs, hydrates or solvates thereof, preferably beclomethasone dipropionate, fluticasone propionate, fluticasone furoate, mometasone furoate, or budesonide.

Alternatively, the active ingredient is a methylxanthine compound. Preferred methylxanthine compounds are selected from the group consisting of theophylline, aminophylline or oxtriphylline and salts, esters, polymorphs, hydrates or solvates thereof.

The use of Long Acting β2-Agonists (LABAs) has long been a key medication to treat the bronchoconstrictive elements of asthma and COPD. Trials have highlighted that the addition of LABAs to the anticholinergic compound ipratropium bromide is more effective than either agent used alone. The combination of a LABA and anticholinergic (preferably a long-acting muscarinic antagonist (LAMA)) is now an important combination therapy for dealing with asthma and COPD.

In a preferred embodiment of the present invention eutectic compositions can be obtained from any combination of LABA and LAMA whereby a specific combination of the LABA and LAMA has maximum interaction between the two crystalline species yielding homogeneity and a single defined melting point.

Specific examples are given for:

| LABA | LAMA |
|---|---|
| Salmeterol xinafoate | Glycopyrronium bromide |
| Formoterol fumarate | Glycopyrronium bromide |
| Indacaterol maleate | Glycopyrronium bromide |
| Salmeterol xinafoate | Tiotropium bromide |
| Formoterol fumarate | Tiotropium bromide |
| SABA | LAMA |
| Fenoterol hydrobromide | Glycopyrronium bromide |
| SABA | SAMA |
| Salbutamol sulphate | Ipratropium bromide |

Particularly preferred combinations of a β2-agonist and an anticholinergic are albuterol and ipratropium bromide, formoterol fumarate and glycopyrronium bromide, salmeterol xinafoate and glycopyrronium bromide, formoterol fumarate and aclidinium bromide, olodaterol and tiotropium bromide, vilanterol and umeclidinium bromide, vilanterol and glycopyrronium bromide, indacaterol maleate and glycopyrronium bromide, salmeterol xinafoate and tiotropium bromide, formoterol fumarate and tiotropium bromide, and, fenoterol hydrobromide and glycopyrronium bromide.

The combination of salmeterol xinafoate (SX) and glycopyrronium bromide (GB) is particularly preferred. It is difficult to dissolve SX and form stable solutions. Dissolving a eutectic composition of SX together with GB allows more highly concentrated, more stable solutions of SX to be formed.

The preferred molar ratio of β2-agonist to anticholinergic is 10:1 to 1:10, preferably 9:1 to 1:9, preferably 4:1 to 1:4, preferably 2:1 to 1:1, preferably 1:1.

The present invention is a method of forming a highly concentrated solution of two pharmaceutically active ingredients. Use of a eutectic composition of active ingredients opposed to a blend enables less solvent to be used, i.e. leads to formation of more highly concentrated solutions. The term “highly concentrated” means a concentration of active which is usually too high to enable the concentrated solution to be used therapeutically for inhalation without being diluted. Thus the first solution generally needs to be diluted and converted into a pharmaceutical preparation before use.

Surprisingly, the concentrated solutions of this invention generally do not require the use any alcohol, excipients, stabilisers or buffering agents to ensure stability. Thus, the concentrated solution preferably does not comprise for instance sodium citrate, sodium hydroxide, hydrochloric acid, sulphuric acid, sodium chloride, calcium chloride, benzalkonium chloride, polysorbate 80, disodium EDTA, sodium phosphate, ethanol, oleic acid or lecithin.

Preferably, the first solvent is a polyol, such as glycerol, polyethylene glycol or propylene glycol.

The first solution may be filtered using techniques conventional in the art.

The first solution may be stored in an ampoule for later use in a nebulizer. Alternatively, the first solution may be diluted with a second solution to form a final solution. The first solution may be too concentrated for medicinal use, or may be highly viscous, such that it cannot be easily volatilised. Typically, the second solvent is selected from water or saline solution.

The method forming a solution of a first and second pharmacologically active ingredient comprising: providing a solid eutectic composition of the first and second pharmacologically active ingredients; providing a first solvent; and dissolving the eutectic composition in the first solvent; can be carried out at room temperature i.e. at 25° C. The method of the present invention can be carried out at a temperature in the range of 15-40° C., preferably, 25-30° C. The method of the present invention can be carried out at a temperature in the range of 15-40° C. and in an inert atmosphere of nitrogen gas i.e. under a nitrogen blanket. One advantage of carrying out the method of the present invention in the above described temperature ranges, and/or under an inert atmosphere, is that discoloration of the solution is reduced. A nitrogen blanket is particularly preferred when the method of the present invention is carried out at 30-40° C.

The pharmaceutical compositions of the present invention can be administered for instance by a nebulizer or a pressurised metered dose inhaler. The invention therefore provides a nebulizer or a pressurized metered-dose inhaler comprising the solution or final solution of the invention. The first or final solution may be stored, for instance, in an ampoule, for later use in a nebuliser. Alternatively the first or final solution may be stored in a PMDI together with a suitable propellant. Nebulizers suitable for use in this invention are disclosed in WO2010/144628. PMDIs suitable for use in this invention are disclosed in WO2011/076843.

The first or final solution may be stored in an ampoule and diluted with a further solvent just before being dispensed in a nebulizer. This further solvent is generally water or saline for nebulizer use. When a nebulizer is used, the solvent used for dissolving the at least two pharmacologically active ingredients must be compatible with the solution used in a nebulizer. Typically, a nebulizer solution is aqueous.

When the solution or final solution is to be used in a PMDI, it is generally mixed with hydrofluoroalkane propellants (e.g. HFA 134 or HFA 227) or a mixture of alcohol and propellants for solution PMDI use. Other diluents suitable for use in solution PMDIs (pressurized metered dose inhalers) may be used.

The concentration of the first active ingredient in the first solution is at least 0.0005% w/w and is preferably in the range 0.0005-1.25% w/w, 0.0005-1% w/w or 0.0005-0.5% w/w, typically 0.005-0.5% w/w, for instance 0.05-0.5% w/w. The concentration of the second active ingredient has, independently, the same preferred ranges. The total active concentration is thus typically in the range 0.001-2.5% w/w, for instance 0.001-1% w/w. The total active concentration is generally considerably higher than the dose to be administered. The skilled person can calculate how much further dilution is required in order to produce a final medicament with the requisite concentration. The first or final solution may be, for instance, diluted 10-20 times to obtain the final medicament.

The first or final solution may further comprise one or more additional pharmaceutically active ingredients selected from β2 agonists, muscarinic antagonists, anticholinergics, corticosteroids, methylxanthine compounds, and salts, esters, polymorphs, hydrates or solvates thereof. Preferably the third pharmaceutically active ingredient, if present, is a corticosteroid, and even more preferably is fluticasone propionate. The further pharmaceutically active ingredient(s) may be added in a solution. The present invention provides solutions comprising of two pharmacologically active ingredients for the treatment of respiratory disease. Preferably the respiratory disease is chronic respiratory disease, preferably, COPD, asthma or cystic fibrosis. The pharmaceutical composition is delivered to the lung by inhalation. Solutions of eutectic compositions are known in the prior art, but have not been disclosed for use in treatment of respiratory diseases.

The respiratory disease may be infection. The infection may be in addition to a chronic respiratory disease such as COPD, asthma or cystic fibrosis, or the infection may be unrelated to a chronic respiratory disease.

The invention makes use of a solid eutectic composition of first and second pharmacologically active ingredients. The formation of these solid eutectic compositions is described further in our previous application published as WO2013/021199. The melting point of the solid eutectic composition depends on the selected combination of a β2-agonist and an anticholinergic, but is generally in the range 50° C. to 175° C., preferably 75-175° C. For example, a 1:1 M GB:SX eutectic mixture has melting onset temperature of 100° C., compared to 190° C. for GB and 124° C. for SX.

Eutectic compositions useful in the present invention comprise two pharmacologically active ingredients in a specific molar or mass ratio which yields a homogenous crystalline solid-solid dispersion characterized with a single melting point and endotherm of melting. The melting point of the eutectic composition is lower than the melting point of either of the pharmacologically active ingredients. Useful eutectic compositions may also comprise more than two pharmacologically active ingredients, such as three or four pharmacologically active ingredients. The melting point of the eutectic composition is lower than the melting point of any of the pharmacologically active ingredients present in the eutectic composition.

In order to determine whether or not a eutectic composition exists or can be found, a person skilled in the art would ordinarily use a number of methods. These are described further in WO2013/021199.

The solid eutectic composition may further comprise an excess of at least one of the pharmacologically active ingredients, wherein the excess forms less than 50 mol % of the amount of the said pharmaceutically active ingredients present in the eutectic composition. That is, if the eutectic composition has a molar ratio of 1:1, in a binary composition of 50 and 50 mol % there will be a zero (0) mol % excess and the mixture will be 100% eutectic composition; in a binary composition of 75 and 25 mol % there will be a 50 mol % excess of the major component [50 mol % eutectic composition]; in a binary composition of 90 and 10 mol % there will be a 80 mol % excess of the major component [20 mol % eutectic composition] and so on. Preferably, the solid eutectic composition may further comprise an excess of at least one of the pharmacologically active ingredients, wherein the excess forms less than 40 mol %, preferably less than 30 mol % of the amount of the said pharmaceutically active ingredients present in the eutectic composition.

The eutectic composition may have a molar ratio of 10:1 to 1:1, preferably 9:1 to 1:1, preferably 4:1 to 1:1, preferably 2:1 to 1:1. The skilled person can determine the eutectic molar ratio of a given combination of pharmacologically active ingredients by DSC. The skilled person can determine the molar ratio of a specific composition comprising two pharmacologically active ingredients. The skilled person can therefore determine the deviation of a given composition from the proportion of the eutectic composition as described by molar excess above.

Thus the solid eutectic composition of the present invention may comprise a mass excess of at least one of the pharmacologically active ingredients. Preferably the excess forms less than 50% by weight, preferably less than 40% by weight, preferably less than 30% by weight of the total weight of the said pharmacologically active ingredients present in the composition. The amount of mass excess of one of the components can be determined by DSC by measuring and integrating the area of the melting endotherm peak corresponding to the excess and measuring and integrating the area of the endotherm peak corresponding to the eutectic composition. Providing the heat of fusion of the excess component in known, the area above and within its respective negative peak as measured in Joules can be converted to a specific molar amount of the excess component.

Preferably at least 90% by weight of at least one of the pharmacologically active ingredients is in the eutectic composition, preferably at least 95% by weight, preferably at least 99% by weight, most preferably substantially all of at least one of the pharmacologically active ingredients is in the eutectic composition. This means that for a given composition, it is preferable that as much of the pharmacologically active ingredients as possible are in the eutectic composition. Preferably when there are two pharmacologically active ingredients, all of one of them is in the form of a eutectic composition, and optionally there is an excess of the other which is not in the form of a eutectic composition.

Where there is an excess of one of the pharmacologically active ingredients, the composition will show eutectic behaviour, that is the melting point of the composition will be reduced compared to the melting point of either of the pharmacologically active ingredients. There may be different melting points for the composition as a whole, that is, part of the composition may have a lower melting point and other parts a higher melting point. The part of the composition with a lower melting point will be the part in a eutectic. The part of the composition with a higher melting point will be an excess of one of the pharmacologically active ingredients. It may be necessary to have an excess of one of the pharmaceutically active ingredients if the therapeutic ratio of the two pharmacologically active ingredients is different to the eutectic ratio. Such a composition is useful in the present invention because the melting point of at least part of the composition is lower than either of the melting point of either of the two pharmacologically active ingredients.

Processes for preparing solid eutectic compositions are described in detail in WO2013/021199.

The invention is now described in the following non-limiting Examples.

### EXAMPLES

### Example 1

### Objective and Experimental Work

The aim of this Example was to determine the solubility of 1:1[M] eutectic mixture of GB:FF (Glycopyrronium bromide and Formoterol fumarate) and GB:SX (Glycopyrronium bromide and salmeterol xinafoate) and to assess the stability of concentrated solutions of GB:FF and GB:SX in selected media. The results are compared to the corresponding blends.

### Experimental Method and Procedures:

### Preparation of 1:1 M GB:SX Eutectic Mixture

Methanol solution of GB/SX was prepared in 1:1 M ratio and added to re-circulating DIPE (diisopropylether) at an addition rate of 0.5 ml/min, solution/non-solvent 1/20 using 40 w US power using Sonolab™ 100 ml ultrasonic vessel based system. Immediate recrystallization and formation of uniform slurry was observed in all cases. Material isolated by spray drying was crystalline as indicated by DSCs.

### Preparation of 1:1 M GB:FF Eutectic Mixture

Methanol solution of GB/FF was prepared in 1:1 M ratio and added to re-circulating TBME (Methyl tert-butyl ether) at an addition rate of 0.5 ml/min, solution/non-solvent 1/20 using 40 w US power using Sonolab™ 100 ml ultrasonic vessel based system. Immediate recrystallization and formation of uniform slurry was observed in all cases. Material isolated by spray drying was crystalline as indicated by DSCs.

Blended sample mixtures in 1:1 M ratio for GB:SX and GB:FF were also prepared to help understand the solubility difference compared to eutectic mixture. A physical blend of the selected active ingredients was prepared by rotational blending (tumble blender) in a glass vial at 50-60 rpm for 5 minutes. Samples of the blended mixture were further used for solubility studies.

The following procedure was used for preparing solubility samples in the polyol medium:
Charge Active Pharmaceutical Ingredients (API) portion-wise (up to 10 mg) to 2 mL of medium in 14 mL glass vial with cap.
Sonicate mixture in 40 KHz ultrasonic bath for up to 5 minutes at 25 to 30 degree. For non-aqueous viscous medium, up to two treatments with sonication were required.
Check for dissolution. Charge more API if dissolution is achieved after sonication.
Repeat API charging and sonication steps until incomplete dissolution is obtained.
Place suspension in 25° C. water bath and hold for at least 30 minutes before proceeding further.
Filter suspension through 0.45 μm syringe filter. Dilute filtrate by volume with

HPLC mobile phase to concentration suitable for HPLC analysis.

For the glycerol sample, 1.5 mg of sample and 3 mL of medium was used.

### Results

### Solubility of 1:1 Molar GB:SX Mixture

| | Drug concentration (ppm) | | | |
|---|---|---|---|---|
| | 1:1 (M)GB:SX | | | |
| Medium | Eutectic Mixture | | Blend | |
| Glycerol | (PXLB083-073-9) | 482 | 254 | (PXLB083-073-13) |
| PEG-400 | (PXLB083-073-10) | 7607 | 1969 | (PXLB083-073-14) |

### Solubility of 1:1 Molar GB:FF Mixture

| | Drug concentration (ppm) | | | |
|---|---|---|---|---|
| | 1:1 (M)GB:FF | | | |
| Medium | Eutectic Mixture | | Blend | |
| Glycerol | (PXLB078-149) | >10000 | 410 | (PXLB083-151-2b) |
| PEG-400 | (PXLB078-149) | >10000 | 1480 | (PXLB083-151-3b) |

Stability testing for 1:1[M] GB:SX eutectic mixture in solution (prepared using PEG-400, stored at 25° C./60% RH) was also carried out by monitoring the change in the content of the active substance after 1 month time point. The concentration of active was determined chromatographically, by analysing the test solutions against freshly prepared standard solutions and monitored against change in ratio over time.

| Batch no. | Solvent | Stability Time-point | ratio |
|---|---|---|---|
| EF14054-1 | PEG400 | Initial | 1.0 |
| | | 1M 25/60 | 1.0 |
| EF14054-2 | PEG400 | Initial | 1.0 |
| | | 1M 25/60 | 1.0 |

### Observations

1:1[M] GB:SX eutectic mixture is more soluble in Glycerol and PEG-400 compared to the 1:1[M] GB:SX blend. Analysis of samples at 1 month time point for PEG-400 solution of 1:1[M] GB:SX eutectic mixture indicates no change in ratio of both the components, i.e. GB and SX appears to reasonable stable. Solubility data of 1:1 M GB:FF combination also indicates that the eutectic mixture has significantly higher solubility compared to the blend sample in the sample ratio.

### Example 2—Calculation of Mole Fraction in SX-GB Formulation

In this invention, for a concentrated solution of eutectic mixture of GB:SX (1:1 molar ratio), the concentrate will contain, 0.5 molar fraction of GB and 0.5 molar fraction of SX.

The mole fraction is moles of target substance divided by total moles involved. In this Example, total moles when 2.5 g (prepared using 1 g of GB and 1.5 g of SX) of 1:1 molar mixture of glycopyrronium bromide (M.W=398.3) and salmeterol xinafoate (M.W=603.74, free base M.W=415.6) is 0.005 (moles of GB=mass of GB/M.W of GB=1/398.3=0.0025 and moles of SX=mass of SX/M.W of SX=1.5/603.74=0.0025, hence total moles=0.0025+0.0025=0.005). Therefore, the mole fraction of GB=0.0025/0.005=0.5 and SX=0.0025/0.005=0.5.

### Example 3—Formation of Solutions for Administration

(1) 10 mg of 1:1 M GB:SX is formulated as a concentrated solution with 1 ml of polyol (PEG400) for storage. For administration by inhalation, the concentrated solution can be diluted with 12.3 g of 2.5% v/v ethanol/HFA 134 solution to give approximately 0.08% w/w of solution. The concentrated solution is 12.5 times more concentrated than the concentration of solution to be administered.

(2) 10 mg of 1:1 M GB:FF is formulated as concentrated solution with 1 ml of polyol (PEG400) for storage. Ampoules for nebulizer use may be filled with solution to desired volume. The solution contained in an ampoule, usually 1 ml, can be mixed with 3-5 ml of water or saline solution into the nebulising chamber of an electro-mechanical nebuliser immediately before the administration to the patient. The concentration of the total active substance concentrate is around 10 mg/ml in PEG400 (0.9% w/w) and 2-3 mg/ml on first dilution, which in turn is around 200-300 times greater than the concentration of the final solution to be administered (typically around 10 mcg/ml).

1. A method of forming a solution of a first and second pharmacologically active ingredient comprising:
providing a solid eutectic composition of the first and second pharmacologically active ingredients;
providing a first solvent; and
dissolving the eutectic composition in the first solvent;
wherein the first and second pharmacologically active ingredients are independently selected from β2 agonists, muscarinic antagonists, anticholinergics, corticosteroids, methylxanthine compounds, and salts, esters, polymorphs, hydrates or solvates thereof.

2. A method according to claim 1, wherein the first pharmacologically active ingredient is a β2 agonist or salt, ester, polymorph, hydrate or solvate thereof, and the second pharmacologically active ingredient is a muscarinic antagonist or salt, ester, polymorph, hydrate or solvate thereof.

3. A method according to claim 2, wherein the β2 agonist is selected from the group consisting of formoterol, salmeterol, carmoterol, indacaterol, vilanterol, arformoterol, bambuterol, isoproterenol, milveterol, clenbuterol, olodaterol, fenoterol, salbutamol, levalbuterol, procaterol, terbutaline, pirbuterol, procaterol, metaproterenol, bitolterol, or ritodrine, albuterol and salts, esters, polymorphs, hydrates or solvates thereof.

4. A method according to claim 2, wherein the muscarinic antagonist is selected from the group consisting of tiotropium, ipratropium, aclidinium, darotropium, glycopyrrolate or umeclidinium and salts, esters, polymorphs, hydrates or solvates thereof.

5. A method according to claim 2, wherein the β2 agonist is a long acting β2 agonist (LABA), preferably selected from the group consisting of salmeterol or formoterol and salts, esters, polymorphs, hydrates or solvates thereof.

6. A method according to claim 2, wherein the muscarinic antagonist is a long acting muscarinic antagonist (LAMA), preferably selected from the group consisting of glycopyrrolate and tiotropium, and salts, esters, polymorphs, hydrates or solvates thereof.

7. A method according to claim 1, wherein the first solvent is a polyol.

8. A method according to claim 7, wherein the polyol is glycerol, ethylene glycol or propylene glycol.

9. A method according to claim 1, wherein the concentration of the first pharmacologically active ingredient in the solution is at least 0.0005% w/w, preferably in the range 0.0005-1% w/w, 0.0005-0.5% w/w, 0.005-0.5% w/w or 0.05-0.5% w/w.

10. A method according to claim 1, wherein the concentration of the second pharmacologically active ingredient in the solution is at least 0.0005% w/w, preferably in the range 0.0005-1% w/w, 0.0005-0.5% w/w, 0.005-0.5% w/w or 0.05-0.5% w/w.

11. A method according to claim 1, wherein the total concentration of pharmacologically active ingredients in the solution is in the range 0.001-2.5% w/w, preferably 0.001-1% w/w.

12. A method according to claim 1, wherein the melting point of the solid eutectic composition is in the range 50-175° C.

13. A method according to claim 1, further comprising filtering the solution.

14. A method according to claim 1, further comprising diluting the solution with a second solvent to obtain a final solution.

15. A method according to claim 1 wherein one or more additional pharmaceutically active ingredients selected from β2 agonists, muscarinic antagonists, anticholinergics, corticosteroids, methylxanthine compounds, and salts, esters, polymorphs, hydrates or solvates thereof is/are added to the solution or final solution.

16. A method according to claim 1 wherein no excipients or stabilisers are added to the solution.

17. A method according to claim 2, further comprising a step of loading the solution or final solution into a pressurised metered dose inhaler or a nebulizer.

18. A solution or final solution obtainable by the method according to claim 1.

19. An ampoule for use with a nebuliser, comprising the solution according to claim 18.

20. (canceled)

21. A pressurised metered dose inhaler comprising the solution according to claim 18 and a propellant.

22. (canceled)

23. (canceled)

24. (canceled)

25. (canceled)

26. A solution of a first and second pharmacologically active ingredient in a first solvent, wherein the first and second pharmacologically active ingredients are independently selected from β2 agonists, muscarinic antagonists, anticholinergics, corticosteroids, methylxanthine compounds, and salts, esters, polymorphs, hydrates or solvates thereof, wherein the concentration of first pharmacologically active ingredient is at least 0.0005% w/w and the concentration of the second pharmacologically active ingredient is at least 0.0005% w/w.

27. A solution according to claim 26 which does not comprise any stabilisers or excipients.

28. A solution according to claim 27 wherein the stabilisers or excipients are selected from sodium citrate, sodium hydroxide, hydrochloric acid, sulphuric acid, sodium chloride, calcium chloride, benzalkonium chloride, polysorbate 80, disodium EDTA, sodium phosphate, ethanol, oleic acid and lecithin.

29. (canceled)

30. (canceled)

31. A final solution obtainable by the method according to claim 14.

32. An ampoule for use with a nebuliser, comprising the final solution according to claim 31.

33. A pressurised metered dose inhaler comprising the final solution according to claim 31 and a propellant.

34. A method of treatment comprising administering to a patient a solution according to claim 18.

35. A method of treatment according to claim 34 wherein the solution is administered to the lung.

36. A method of treatment according to claim 34 wherein the treatment is of respiratory disease, preferably wherein the respiratory disease is chronic respiratory disease, preferably COPD, asthma or cystic fibrosis.

37. A method of treatment comprising administering to a patient a final solution according to claim 31.

38. A method of treatment according to claim 36, comprising use of an ampoule with a nebuliser, wherein the ampoule comprises a final solution obtainable by the following method:
providing a solid eutectic composition of the first and second pharmacologically active ingredients;
providing a first solvent;
dissolving the eutectic composition in the first solvent; and
diluting the solution with a second solvent to obtain a final solution,
wherein the first and second pharmacologically active ingredients are independently selected from β2 agonists, muscarinic antagonists, anticholinergics, corticosteroids, methylxanthine compounds, and salts, esters, polymorphs, hydrates or solvates thereof,
and wherein the final solution is diluted by a third solvent prior to administration to the patient, preferably by administration to the lung.

39. A method according to claim 14, wherein one or more additional pharmaceutically active ingredients selected from β2 agonists, muscarinic antagonists, anticholinergics, corticosteroids, methylxanthine compounds, and salts, esters, polymorphs, hydrates or solvates thereof is/are added to the final solution.

40. A method according to claim 14, further comprising a step of loading the final solution into a pressurised metered dose inhaler or a nebulizer.
